(19) United States
(12) Patent Application Publication
(10) Pub. No.: US 2024/0252305 A1
(43) Pub. Date: Aug. 1, 2024

(54) STENT WITH SELECTIVE MEMBRANE COATING

(71) Applicant: BOSTON SCIENTIFIC SCIMED, INC., Maple Grove, MN (US)

(72) Inventor: Martyn G. Folan, Galway (IE)

(73) Assignee: BOSTON SCIENTIFIC SCIMED, INC., Maple Grove, MN (US)

(21) Appl. No.: 18/421,461

(22) Filed: Jan. 24, 2024

Related U.S. Application Data
(60) Provisional application No. 63/481,671, filed on Jan. 26, 2023.

(51) Int. Cl. A61F 2/04; A61F 2/00

(52) U.S. Cl. CPC A61F 2/04; A61F 2/0077; A61F 2002/0081; A61F 2002/041; A61F 2220/0008

(57) ABSTRACT

Medical devices and methods for using medical devices are disclosed. An example expandable medical device includes a tubular scaffold including an inner surface, an outer surface, a proximal end region, a distal end region, and a medial region extending between the proximal end region and the distal end region, wherein the tubular scaffold defines a folded-over portion, and wherein the folded-over portion extends from the distal end region toward the proximal end region. Further, the medical device includes a membrane disposed along the at least a portion of the medial region of the tubular scaffold.

### CROSS-REFERENCE TO RELATED APPLICATIONS

This application claims the benefit of priority under 35 U.S.C. § 119 of U.S. Provisional Application No. 63/481,671, filed Jan. 26, 2023, the entire disclosure of which is hereby incorporated by reference.

### TECHNICAL FIELD

The present disclosure pertains to medical devices, methods for manufacturing medical devices, and uses thereof. More particularly, the present disclosure pertains to stents having selective membrane coatings for implantation in body lumens, and associated methods.

### BACKGROUND

Implantable medical devices (e.g., expandable stents) may be designed to treat a variety of medical conditions in the body. For example, some expandable stents may be designed to radially expand and support a body lumen and/or provide a fluid pathway for digested material, blood, or other fluid to flow therethrough following a medical procedure. Some medical devices may include radially or self-expanding stents which may be implanted transluminally via a variety of medical device delivery systems. These stents may be implanted in a variety of body lumens such as coronary or peripheral arteries, the esophageal tract, gastrointestinal tract (including the intestine, stomach and the colon), tracheobronchial tract, urinary tract, biliary tract, vascular system, etc.

In some instances it may be desirable to design stents to include sufficient flexibility while maintaining sufficient radial force to open the body lumen at the treatment site. However, in some stents, the compressible and flexible properties that assist in stent delivery may also cause a stent to migrate from its originally deployed position. For example, stents that are designed to be positioned in the gastrointestinal and/or biliary tract may migrate due to peristalsis (i.e., the involuntary constriction and relaxation of the muscles of the stomach, intestine, and colon). Further, the generally moist and inherently lubricious environment of the stomach, intestine, colon, etc. may contribute to a stent's tendency to migrate when deployed therein. Further yet, the relative motion of non-connected structures (e.g., the relative motion of a hepatic duct and the stomach) may contribute to a stent's tendency to migrate when deployed therein.

Various medical procedures involve the temporary or permanent joining of non-connected anatomical structures. Some examples include a hepaticogastrostomy (HGS), which involves joining a hepatic duct and the stomach to drain the bile duct, EUS-guided gallbladder drainage (EUS-GBD), utilized for the treatment of acute cholecystitis and symptomatic cholelithiasis in patients who are poor operative candidates, a gastrojejunal(GJ) bypass or gastrojejunostomy procedure to create an anastomosis between the small intestine and stomach wall, and stomas to create an artificial opening into the large intestine or other region of the digestive tract. In these medical procedures, peristalsis and gross organ movement in one or both of the anatomical structures being connected may create difficulties in using stents to join the structures due to stent migration. Accordingly procedures may require a stent to permit leak-free drainage from one anatomical structure (e.g., hepatic ducts) to another anatomical structure (e.g., stomach) while also permitting tissue ingrowth into the stent to prevent stent migration.

Therefore, it may be desirable to design a stent having both drainage capabilities and anti-migration features to reduce the stent's tendency to migrate. Examples of medical devices including both drainage and anti-migration features, and methods of using them are disclosed herein.

### SUMMARY

This disclosure provides design, material, manufacturing method, and use alternatives for medical devices. An example expandable medical device includes a tubular scaffold including an inner surface, an outer surface, a proximal end region, a distal end region, and a medial region extending between the proximal end region and the distal end region, wherein the tubular scaffold defines a folded-over portion, and wherein the folded-over portion extends from the distal end region toward the proximal end region. Further, the medical device includes a membrane disposed along the at least a portion of the medial region of the tubular scaffold.

Alternatively or additionally to the embodiment above, wherein, wherein the folded-over portion extends away from the outer surface of the medial region toward the proximal end region at an acute angle.

Alternatively or additionally to the embodiment above, wherein the folded-over portion includes interstices extending from an outer surface of the folded-over portion to an inner surface of the folded-over portion, and wherein the interstices are configured to permit tissue to grow therein.

Alternatively or additionally to the embodiment above, wherein the folded-over portion extends circumferentially around a longitudinal axis of the tubular scaffold.

Alternatively or additionally to the embodiment above, wherein the folded-over portion is positioned substantially parallel to a central longitudinal axis of the stent.

Alternatively or additionally to the embodiment above, wherein the membrane extends along an inner surface of the medial region of the tubular scaffold from the distal end region to the proximal end region.

Alternatively or additionally to the embodiment above, wherein the membrane extends along a portion of an inner surface of the medial region of the tubular scaffold, and wherein a portion of the medial region is devoid of the membrane.

Alternatively or additionally to the embodiment above, wherein the portion of the medial region which is devoid of the membrane is positioned radially interior of to the folded-over portion.

Alternatively or additionally to the embodiment above, wherein the membrane is configured to maintain a passageway for fluid to flow therethrough.

Alternatively or additionally to the embodiment above, wherein the membrane is formed from an elastic material.

Alternatively or additionally to the embodiment above, wherein the medical device further comprises a retention member extending radially away from the outer surface at the proximal end region, wherein the retention member has a distally facing surface positioned substantially parallel to a proximally facing surface.

Alternatively or additionally to the embodiment above, wherein the tubular scaffold includes interstices extending from the outer surface of the tubular scaffold to the inner surface of the tubular scaffold, and wherein the membrane spans the interstices of the portion of the tubular scaffold which defines the retention member.

Alternatively or additionally to the embodiment above, wherein the membrane is in direct contact with the inner surface of the portion of the tubular scaffold defining the retention member.

Alternatively or additionally to the embodiment above, wherein the distal end region of the tubular scaffold further includes a flared portion.

Alternatively or additionally to the embodiment above, wherein the folded-over portion is spaced apart from and extends away from the outer surface of the flared portion at an acute angle.

Alternatively or additionally to the embodiment above, wherein the flared portion includes interstices extending from the outer surface of the tubular scaffold to the inner surface of the tubular scaffold, wherein the flared portion is devoid of the membrane such that tissue is permitted to grow through the interstices of the tubular scaffold along the flared portion.

Alternatively or additionally to the embodiment above, wherein the retention member has an outermost diameter, wherein the flared portion has an outermost diameter, wherein the folded-over portion has an outermost diameter, and wherein the outmost diameter of the retention member is greater than the outmost diameter of the flared portion, the folded-over portion or both the flared portion and the folded-over portion.

Another example expandable medical device includes a tubular scaffold including an inner surface, an outer surface, a proximal end region, a distal end region, and a medial region extending between the proximal end region and the distal end region, wherein the tubular scaffold defines an everted portion, and wherein the everted portion extends from the distal end region toward the proximal end region. The medical device also includes a membrane disposed along a first portion of the medial region of the tubular scaffold, wherein a second portion of the medial region of the tubular scaffold is devoid of the membrane, and wherein the second portion of the medial region of the tubular scaffold which is devoid of the membrane is positioned radially interior to the everted portion.

Alternatively or additionally to the embodiment above, wherein the everted portion extends away from the outer surface of the medial region at an acute angle.

Another expandable medical device includes a tubular scaffold including an inner surface, an outer surface, a proximal end region, a distal end region, and a medial region extending between the proximal end region and the distal end region, wherein the tubular scaffold defines a folded-over portion, and wherein the folded-over portion extends from the distal end region toward the proximal end region. The medical device also includes a membrane disposed along a first portion of the medial region of the tubular scaffold, wherein a second portion of the medial region of the tubular scaffold is devoid of the membrane, and wherein the second portion of the medial region of the tubular scaffold which is devoid of the membrane is positioned radially interior to and spaced apart from the folded-over portion. Further, the tubular scaffold further comprises a retention member extending radially away from the medial region, wherein the retention member is a double-walled flange having a distal wall positioned substantially parallel to and spaced apart from a proximal wall of the double-walled flange.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates a region of the digestive tract;

FIG. 2 illustrates an example stent including an inner membrane and a folded-over portion;

FIG. 3 is a cross-sectional view of the example stent taken along line 3-3 of FIG. 2;

FIG. 4 is an alternative cross-sectional view of the stent of FIG. 2 including an inner membrane;

FIG. 5 is an alternative cross-sectional view of the stent of FIG. 2 including an inner membrane;

FIG. 6 illustrates an example stent including an inner membrane and a folded-over portion;

FIG. 7 is a cross-sectional view of the example stent taken along line 7-7 of FIG. 6;

FIG. 8 is an alternative cross-sectional view of the stent of FIG. 6 including an inner membrane;

FIG. 9 is an alternative perspective view of an example stent;

FIG. 10 illustrates an example stent positioned in a portion of the digestive tract.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

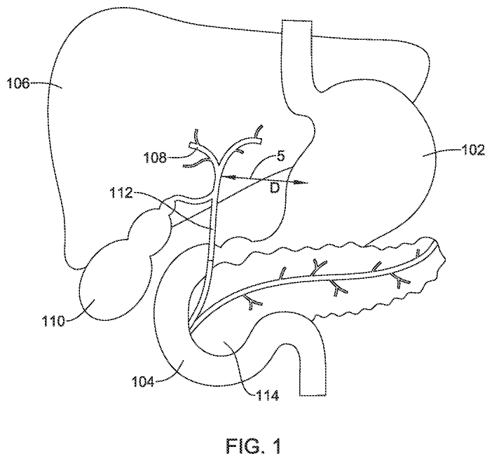

FIG. 1 illustrates various organs in the digestive tract, including the stomach 102, duodenum 104, liver 106, hepatic duct 108, gallbladder 110, common bile duct 112, and pancreas 114.

Bile, which is produced in the liver 106, flows through a series of hepatic ducts 108 that drain into one large duct called the common bile duct (CBD) 112. The CBD then connects to the duodenum 104, allowing the bile to flow into the duodenum for digestion. If the hepatic or bile ducts become blocked, bile cannot drain normally and backs up or builds up in the liver 106. Blocked bile ducts and the resulting increase in bile pressure may cause abdominal pain, jaundice, dark urine, nausea and poor appetite, leading to potentially serious conditions.

Endoscopic retrograde cholangiopancreatography (ERCP) may be used to diagnose and treat conditions of the bile ducts, including, for example, gallstones, inflammatory strictures, leaks (e.g., from trauma, surgery, etc.), and cancer. Blockage of the biliary duct may occur in many of the disorders of the biliary system, including the disorders of the liver, such as, primary schlerosing cholangitis, stone formation, scarring in the duct, etc. Draining blocked fluids from the biliary system may be used to treat the disorders. Methods of biliary drainage include the placement of plastic or metal stents to relieve the blockage. In the case of a gallstone causing the obstruction in the duct, a number of products are also available to resolve this through ERCP. However, access to the bile ducts via ERCP may not be possible due to a variety of reasons such as a tumor blocking the passageway, anatomic variation, periampullary diverticula, post ampullary removal surgery (e.g., a Whipple procedure), etc. When ERCP methods prove unsuccessful, percutaneous drainage (PTCD) can be performed. However, PTCD may be associated with complications such as bleeding and bile leakage. If subsequent internal drainage cannot be achieved, the patient would have to accept long-term external biliary drainage, which can be uncomfortable and have significant impairment of quality of life.

Endoscopic Ultrasound (EUS) guided biliary drainage (BD) offers an alternative option to surgery and percutaneous drainage for treating obstructive jaundice when ERCP drainage fails. Hepaticogastrostomy (HGS) may be performed to join the hepatic duct 108 to the stomach 102. This would allow the build-up of bile to flow into the stomach and may relieve the symptoms caused by bile buildup, i.e. jaundice. However, the hepatic duct 108 and the stomach 102 are spaced apart by a distance D indicated by the arrow 5 in FIG. 1. The distance D between the organs may require a relatively long stent. Additionally, as the stomach muscles contract to churn food, the distance D between the gastric wall of the stomach 102 relative to the hepatic duct 108 varies, from a relatively small distance D when the stomach is relaxed as to a greater distance when the stomach is contracted. In addition to the gastric wall flexing, the stomach undergoes peristalsis during digestion. This relative motility of the stomach is understood to be complex, in three dimensions rather than in an exclusively linear manner. The distance between target organs to be joined, the relative movement of at least one of the organs, as well as the normal motion of the body (e.g., twisting, running, jumping, etc.) may increase the chance of stent migration.

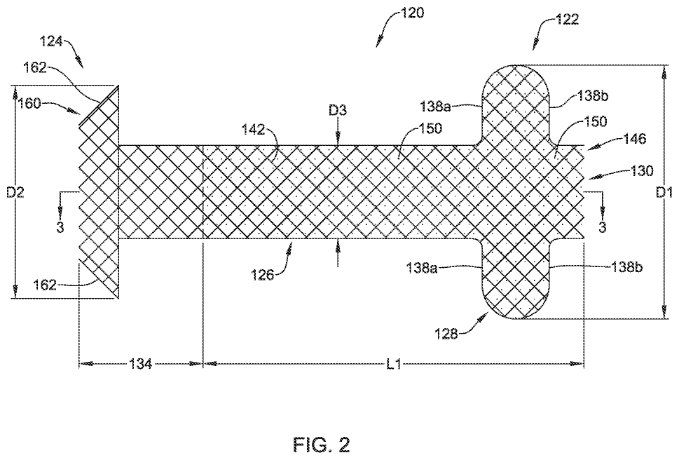

FIG. 2 illustrates an example expandable medical device, namely a stent 120 (e.g., a drainage stent) including a first end region 122 (e.g., a proximal end region), a second end region 124 (e.g., distal end region) and a medial region 126 extending between the first end region 122 and the second end region 124. The stent 120 may include one or more stent strut members 142 forming a tubular scaffold. Stent strut members 142 may extend helically, longitudinally, circumferentially, or otherwise along stent 120. While FIG. 2 shows stent strut members 142 extending along the entire length of stent 120, in other examples, the stent strut members 142 may extend only along a portion of stent 120.

In some examples, the stent 120 may be a self-expanding stent. Self-expanding stent examples may include stents having an expandable scaffold. In some instances, the self-expanding stent may have one or more filaments 142 combined to form a rigid and/or semi-rigid tubular stent scaffold. For example, the stent filaments 142 of the stent 120 may include wires or filaments which are braided, wrapped, intertwined, interwoven, weaved, knitted, looped (e.g., bobbinet-style) or the like to form the tubular scaffold. For example, while the example stents disclosed herein may resemble a braided stent, this is not intended to limit the possible stent configurations. Rather, the stents depicted in the Figures may be stents that are braided, knitted, wrapped, intertwined, interwoven, weaved, looped (e.g., bobbinet-style) or the like to form the stent scaffold. In various embodiments, the woven, braided and/or knitted member(s) may include a single filament woven upon itself, or multiple filaments woven together. In various embodiments, any of the woven, braided and/or knitted member(s), which comprise the elongate tubular body, may include a variety of different cross-sectional shapes (e.g., oval, round, flat, square, etc.).

Alternatively, the stent 120 may be a monolithic structure formed from a cylindrical tubular member, such as a single, cylindrical tubular laser-cut Nitinol tubular member, in which the remaining portions of the tubular member form the tubular scaffold. Openings or interstices through the wall of the stent 120 may be defined between adjacent filaments 142 or struts of the tubular scaffold.

The stent 120 in the examples disclosed herein may be constructed from a variety of materials. For example, the stent 120 (e.g., self-expanding or balloon expandable) may be constructed from a metal (e.g., Nitinol, Elgiloy, etc.). In other instances, the stent 120 may be constructed from a polymeric material (e.g., PET). In yet other instances, the stent 120 may be constructed from a combination of metallic and polymeric materials. Additionally, stent 120 may include a bioabsorbable and/or biodegradable material.

Additionally, the stent 120 may be configured to shift between a first (e.g., constrained, collapsed, non-expanded) configuration and a second (e.g., non-constrained, expanded) configuration. In an expanded configuration, the first end region 122 of the stent 120 may include a retention member 128 defining a first opening 130. The retention member 128 may be formed from the stent strut members 142 used to form other portions of the stent 120. For example, the retention member 128 may be formed from the same stent strut members 142 used to form the medial region 126.

FIG. 2 further illustrates that, in some examples, the medial region 126 may include a uniform outer diameter D3 along its length. Additionally, the medial region 126 of the stent 120 may include a circumference and a longitudinal axis. The medial region 126 of the stent 120 may extend between the second end region 124 and the retention member 128 of the first end region 122. The stent 120 may define an open interior lumen (e.g., passage, channel, etc.) extending from the first end region 122 to the second end region 124.

The retention member 128 may extend radially away from (e.g., substantially perpendicular to) the longitudinal axis of the medial region 126 to define a first surface 138a and a second surface 138b. In some instances, the first surface 138a, which may be a distally facing surface of the retention member 128, is substantially parallel to the second surface 138b, which may be a proximally facing surface of the retention member 128. The first surface 138a may be configured to atraumatically engage a (e.g., inner) tissue wall of a first body lumen (e.g., the stomach or duodenum).

FIG. 2 further illustrates that the second end region 124 of the stent 120 may be everted on itself to form a folded-over portion 160. For example, it can be appreciated that to form the folded-over portion 160, the stent scaffold may be wrapped, intertwined, interwoven, weaved, looped such that the individual filaments 142 extend continuously along the medial region 126 to the second end region 124 of the stent 120 whereby they curve and extend back (e.g., bend back) toward the first end region 122 of the stent 120. In the folded-over configuration shown in FIG. 2, the folded-over portion 160 includes a first outwardly-facing surface 162. It can be appreciated that this first outwardly facing surface 162 may extend circumferentially around the longitudinal axis of the stent 120. Additionally, FIG. 2 illustrates that the folded-over portion 160 may have a maximum outer diameter D2.

Further, the outwardly facing surface 162 of the folded-back portion 160 may be configured to atraumatically engage a (e.g., inner) tissue wall of an adjacent or apposed second body lumen (e.g., a biliary duct). In the example stent 120 illustrated in FIG. 2, the outwardly facing surface 162, the first surface 138a and the second surface 138b may prevent or limit movement/migration of the deployed stent 120 within or between the first and second body lumens.

FIG. 2 further illustrates that, in some examples, an outer diameter D1 of the retention member 128 may be greater than the outer diameter D2 of the folded-over portion 160. However, in other examples, the outer diameter D1 of the retention member 128 may be equal to the outer diameter D2 of the folded-over portion 160. The medial region 126 may include a constant outer diameter D3 extending between the second end region 124 and the retention member 128, whereby the diameter D3 of the medial region 126 is less than the diameters D1 and D2 of the retention member 128 and the folded-over portion 160, respectively.

In some examples, the diameter D1 may be about 5 mm to about 40 mm, or about 10 mm to about 35 mm, or about 15 mm to about 30 mm, or about 20 mm to about 25 mm, or about 10 mm to about 20 mm, or about 20 mm to about 35 mm. In some examples, the diameter D2 may be about 5 mm to about 40 mm, or about 10 mm to about 35 mm, or about 15 mm to about 30 mm, or about 20 mm to about 25 mm, or about 10 mm to about 20 mm, or about 20 mm to about 35 mm. In some examples, the diameter D3 may be about 2 mm to about 20 mm, or about 4 mm to about 18 mm, or about 6 mm to about 14 mm, or about 8 mm to about 12 mm, or about 15 mm to about 25 mm.

Additionally, in some examples, the first end region 122 of the stent 120 may include free ends 146 of the one or more woven, braided or knitted strut members 142 which are not connected, and instead form sharp or pointed free ends of the strut members 142. As will be understood by those of skill in the art, the surface 138a of the retention member 128 may atraumatically engage an inner tissue wall of a first body lumen such that the free ends 146 extend into the first body lumen and do not contact the tissue wall.

As discussed herein, for HGS patients, stent migration may cause serious complications including death. If there is no tissue ingrowth-based adhesion at various anatomical regions, a deployed stent may migrate proximally into the stomach, causing leakage of biliary contents into the peritoneum, resulting in peritonitis. If there is sufficient or excessive adhesion at the hepatic duct, the stent may migrate distally into the peritoneum, causing leakage of biliary and stomach contents into the peritoneum, also causing peritonitis. Additionally, a migrated stent is free to abrade the outer gastric wall and other organs or vessels in the vicinity. Anatomically, stent migration can occur as a result of the hepatic duct being a generally static vessel, whereas the stomach is a highly motile vessel, as discussed herein. Accordingly, one method to reduce stent migration may include exposing bare metal portions of the stent to the tissue of the body lumen. The stent scaffold may then provide a structure that promotes tissue ingrowth (e.g., a hyperplastic response) into the interstices or openings thereof. The tissue ingrowth may anchor the stent in place and reduce the risk of stent migration.

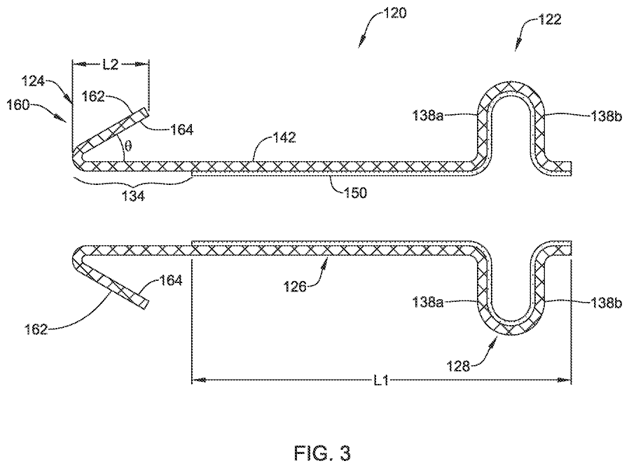

FIG. 2 illustrates that, in some examples, the tubular scaffold of the stent 120 may include one or more non-covered (e.g., bare) portions designed to promote tissue ingrowth (e.g., a hyperplastic response) into the interstices or openings thereof. The non-covered portions may be devoid of a membrane, coating or other covering and thus the interstices of the tubular scaffold in the non-covered portions are open to tissue ingrowth. For example, FIG. 2 illustrates that the stent 120 may include the non-covered portion 134. It can be appreciated that, in some examples, the non-covered portion 134 of the tubular scaffold of the stent 120 may include the folded-over portion 160 of the stent 120. In other words, the folded-over portion 160 of the stent 120 shown in FIG. 2 may be bare (e.g., devoid or free of a membrane, coating, etc.) allowing tissue ingrowth through the interstices of the tubular scaffold along the folded-over portion 160. Further, as will be discussed in greater detail with respect to FIG. 3, the non-covered portion 134 of the tubular scaffold of the stent 120 may include a portion of the medial region 126 of the stent 120 proximate the folded-over portion 160. It can be appreciated that when positioned in a body lumen, the outer-facing surface 162 of the folded-over portion 160, which may be a non-covered or bare portion of the stent 120, may contact the vessel wall.

Additionally, FIG. 2 illustrates that the stent 120 may include a membrane 150 (e.g., coating, membrane coating, etc.) extending along the tubular scaffold of the stent 120, such as within the lumen of the tubular scaffold of the stent 120. For example, FIG. 2 illustrates that the membrane 150 may extend along a length L1 of the tubular scaffold of the stent 120. The length L1 along which the membrane 150 extends within the lumen of the tubular scaffold of the stent 120 may include a portion of the medial region 126 and the first end region 122 (including the retention member 128). As will be discussed in greater detail below, the membrane 150 may extend along an inner surface and/or outer surface of the filaments 142 forming the medial region 126 in order to span across the interstices of the medial region 126, whereby the membrane 150 defines a leak-free, passageway (e.g., channel, lumen, tunnel, etc.) which may permit drainage of bodily substances (e.g., bile) from one anatomical organ (e.g., liver) to another anatomical organ (e.g., stomach). In some examples, the membrane 150 may be an elastomeric or non-elastomeric material. For example, the membrane 150 may be a polymeric material, such as silicone, polyurethane, UE, PVDF, PTFE, ePTFE, ChronoFlex® or similar biocompatible polymeric formulations.

FIG. 3 illustrates a cross-sectional view of the stent 120 taken along line 3-3 of FIG. 2. FIG. 3 illustrates the first end region 122, the second end region 124 and the medial region 126. The stent 120 may be formed from one or more braided, knitted, wrapped, intertwined, interwoven, weaved, looped (e.g., bobbinet-style) filaments 142 to form the tubular scaffold of the stent 120. FIG. 3 also illustrates the folded-over portion 160 positioned along the second end region 124. The folded-over portion 160 may include the first outer facing surface 162 described herein. FIG. 3 further illustrates that the folded-over portion 160 may also include a second inward facing surface 164. The inward facing surface 164 may be defined as the surface of the folded-over portion 160 which faces toward the longitudinal axis of the stent 120, and thus faces toward the medial region 126 of the stent 120. Additionally, the stent may include a retention member 128 positioned along the first end region 122.

FIG. 3 further illustrates the folded-over portion 160 extending away from an outer surface of the medial region 126 at an angle θ. In some instances, the angle θ may be about 20 degrees or more, 30 degrees or more, 45 degrees or more, 60 degrees or more, or 70 degrees or more. The folded-over portion 160 may taper radially outward as the folded-over portion 160 extends toward the first end region 122 of the stent 120. Thus, the folded-over portion 160 may taper radially outward as the folded-over portion 160 extends over and surrounds a portion of the medial region 126. Further, FIG. 3 illustrates the folded-over portion 160 extending along the longitudinal axis of the stent 120 for a length L2. It can be appreciated that the stent 120 may include embodiments in which the folded-over portion 160 extends away from the outer surface of the medial region 126 at a variety of different angles θ. Further, it can be appreciated that the stent 120 may include embodiments in which the folded-over portion 160 extends along the medial region 126 at a variety of different lengths.

FIG. 3 further illustrates the membrane 150 extending along a portion of the inner surface of the filaments 142 defining the tubular scaffold of the stent 120. In other words, FIG. 3 illustrates that the stent 120 may include the membrane 150 extending within the inner lumen of the tubular scaffold of the stent 120. In other instances, the membrane 150 may extend along a portion of the outer surface of the filaments 142 defining the tubular scaffold of the stent 120. As discussed herein, FIG. 3 illustrates the membrane extending along a length L1 of the medial region 126 of the stent 120, occluding the interstices between the filaments 142 along the medial region 126.

As discussed herein, the first outer-facing surface 162 and the second inner-facing surface 164 of the folded-over portion 160 may be devoid of the membrane 150, thereby permitting tissue ingrowth (e.g., a hyperplastic response) into the interstices or openings thereof. Additionally, FIG. 3 illustrates that a portion 134 of the medial region 126 may be devoid of the membrane 150, thereby permitting tissue ingrowth (e.g., a hyperplastic response) into the interstices or openings thereof. The non-covered portion 134 may extend from the covered portion of the medial region 126 to the folded-over portion 160. The entire non-covered portion 134 may be devoid of the membrane 150, coating or other covering and thus the interstices of the tubular scaffold in the non-covered portion 134 may be open to tissue ingrowth.

It can be appreciated that as the proportion of the stent 120 which includes a membrane 150 increases, the proportion of the stent 120 which does not include a membrane decreases, and vice versa. In some examples, the ratio of the covered portion L1 to the non-covered portion 134 may be about 9:1 of the covered portion L1 to the uncovered portion 134, or about 4:1 of the covered portion L1 to the uncovered portion 134, or about 7:3 of the covered portion L1 to the uncovered portion 134, or about 3:2 of the covered portion L1 to the uncovered portion 134, or about 1:1 of the covered portion L1 to the uncovered portion 134. The uncovered portion 134 may be designed to be more flexible than the covered portion L1, allowing for better response when positioned in highly motile regions of the body.

Further, it can be appreciated that designing the stent 120 to include a relatively greater length of the uncovered portion 134 to the covered portion L1 may dedicate a greater percentage of the overall stent length to bare region duct drainage and anti-migration features. However, while longer uncovered portions 134 may allow for higher potential for side branch drainage and anti-migration ingrowth, the reduction in the covered portion L1 may reduce the effective length of the stent 120 that can be bridged between disconnected anatomical vessels (e.g., the distance between the gastric wall into the liver parenchyma). The uncovered portion 134 and the length L2 of the folded-over portion 160 must be sized to allow enough resistance to stent migration (so as to prevent migration initially through mechanical resistance and eventually with the addition of tissue ingrowth) while also allowing the stent 120 to include enough of a covered portion L1 to bridge the distance between disconnected anatomical vessels (e.g., the distance between the gastric wall into the liver parenchyma).

It can be appreciated that tissue may be permitted to grow around, between, through, within, etc. those strut members 142 of the tubular scaffold of the stent 120 in which the membrane 150 is not attached. In other words, FIG. 3 illustrates multiple “tissue ingrowth regions” defined along both the folded-over portion 160 and along the non-covered portion 134 along the medial region 126 of the tubular scaffold of the stent 120. The uncovered or bare portions of the tubular scaffold of the stent 120 may provide structures that promote tissue ingrowth to anchor the stent 120 in place and reduce the risk of stent migration. Further, it can be appreciated that the membrane 150 may define a leak-free, passageway (e.g., channel, lumen, tunnel, etc.) which may permit drainage of bodily substances (e.g., bile) from one anatomical organ (e.g., liver) to another anatomical organ (e.g., stomach).

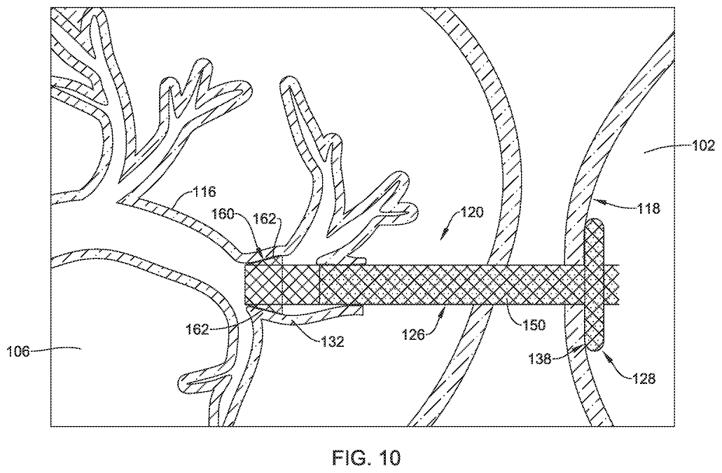

When positioned in the body (e.g., FIG. 10 illustrates the stent 120 positioned in a hepatic duct 116 of the liver 106), the outer facing surface 162 of the folded-over portion 160 may directly contact the vessel wall. Accordingly, the non-covered filaments 142 forming the folded-over portion 160 may promote an initial (primary) tissue ingrowth region to anchor the stent 120 in place and reduce the risk of stent migration. In other words, after the initial placement of the stent 120 in the body lumen, the interstices formed from the filaments 142 of the folded-over portion 160 may provided openings through which tissue ingrowth may occur. This may be the primary mechanism for which the distal end region 124 of the stent 120 may be anchored to the duct.

However, it can be appreciated that the non-covered portion 134 may also provide a secondary tissue ingrowth region to anchor the stent 120 in place and reduce the risk of stent migration. For example, tissue may initially grow within the folded-over portion 160, during which time the non-covered portion 134 may remain bare, thereby allowing fluid drainage therethrough into membrane 150 covered portion of the medial region 126. However, over time, tissue may grow into the interstices of the non-covered portion 134, thereby providing a secondary anchoring tissue ingrowth region. As tissue ingrowth occurs within the non-covered portion 134, the ability for fluid drainage to occur within the non-covered portion 134 may decrease.

It can be further appreciated that the folded-over portion 160 may provide a radially outward force on the vessel wall within which the stent 120 is positioned. In other words, the folded-over portion may include a spring force which forces the outer facing surface 162 of the folded-over portion 160 to oppose the tissue of the vessel wall. Further, this radially outward spring force also acts to create a space between the inner facing surface 164 of the folded-over portion 160 and the outer surface of the non-covered portion 134 of the medial region 126. It can be appreciated that this design may create a double braided layer of bare, non-covered filaments (e.g., one layer of the folded-over portion 160 positioned radially outward of a second layer, which is the bare non-covered portion 134 of the medial region 126).

It can further be appreciated that the relative amount of non-covered (e.g., bare) portions of the stent 120 versus the membrane 150 covered portions may be customized (e.g., tailored) based on the where in the body the stent 120 may be positioned. For example, tissue ingrowth may be relatively fast in some organs (e.g., the hepatic duct) and, therefore, it may be desirable to design the stent 120 to have a shorter folded-over portion 160 (e.g., a shorter length L2 of the outer facing surface 162 in FIG. 3) for primary tissue ingrowth and anchorage. It can be appreciated that designing the stent 120 to include a shorter length L2 of the outer facing surface 162 of the folded over region 160 may permit more of the non-covered portion 134 to drain fluid from side-branches of the organ in which the stent 120 is positioned.

Additionally, tissue ingrowth may be relatively slow in some organs (e.g., the pancreas) and, therefore, it may be desirable to design the stent 120 to have a longer folded-over portion 160 (e.g., a longer length L2 of the outer facing surface 162 in FIG. 3) for primary tissue ingrowth and anchorage. It can be appreciated that designing the stent 120 to include a longer length L2 of the outer facing surface 162 of the folded-over region 160 may result in more tissue ingrowth along the outer facing surface 162 of the folded-over portion 160 to help anchor the stent 120 in place while continuing to provide side-branch drainage.

One advantage of the stent designs described herein which include a folded-over portion and a non-covered medial portion is that the non-covered medial portion (e.g., portion 134 in FIG. 3) and the folded-over portion (e.g., folded-over portion 160 in FIG. 3) allow for side branch drainage and tissue ingrowth, respectively, to be achieved independent of one another with the promotion of tissue ingrowth to have less impact on the prolonged drainage of side branch vessels. This design is combined with the membrane 150 which may define a leak-free, passageway (e.g., channel, lumen, tunnel, etc.) which may permit drainage of bodily substances (e.g., bile) from one anatomical organ (e.g., liver) to another anatomical organ (e.g., stomach).

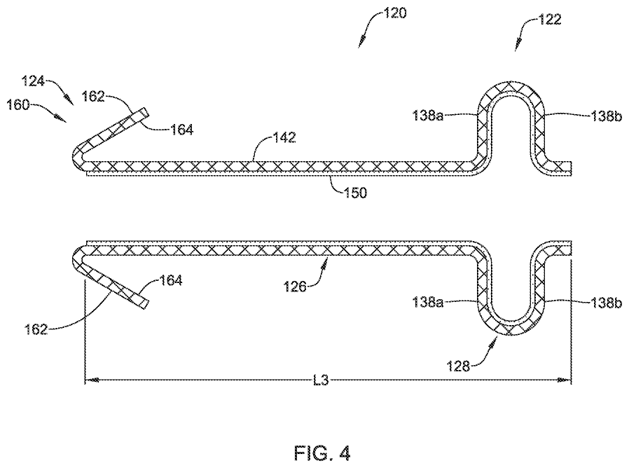

It can be further appreciated that the relative lengths of the non-covered portion 134 and the covered portion L1 may differ from that illustrated in FIG. 3. For example, FIG. 4 illustrates a cross-sectional view of the stent 120 identical to FIG. 3 except that more of the stent 120 may include a membrane 150. FIG. 4 illustrates the membrane 150 extending along substantially the entire length of the medial region 126 to the second end region 124 (e.g., FIG. 4 illustrates the entire length of the medial region 126 may include the membrane 150). The portion of the medial region 126 which includes the membrane is identified as length L3 in FIG. 4. However, FIG. 4 further illustrates that the membrane 150 may extend along the medial region 126 to the point at with the stent scaffold folds over upon itself (e.g., the point at which the stent struts are everted to form the folded-over portion 160). While FIG. 4 illustrates the membrane 150 lengthening from length L1 (shown in FIG. 3) to length L3 (shown in FIG. 4), the folded-over portion 160 may remain devoid of the membrane 150.

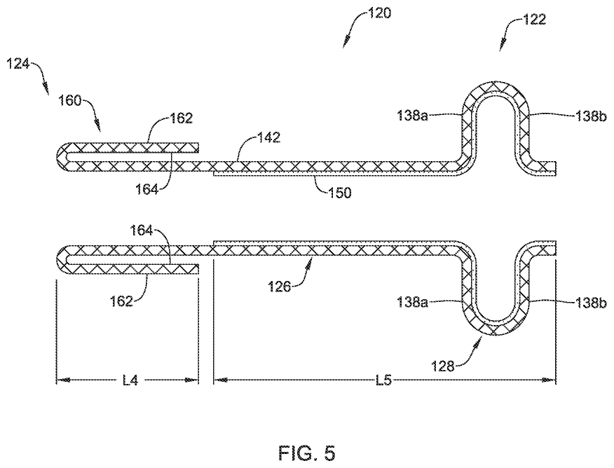

For example, FIG. 5 illustrates an alternative embodiment of the stent 120 whereby the folded-over portion 160 has been everted approximately 180 degrees such that the outer facing surface 162 and the inner facing surface 164 may be substantially parallel to the longitudinal axis of the stent 120. Additionally, FIG. 5 illustrates the folded-over portion 160 extending along the medial region 126 of the stent 120 a length L4. FIG. 5 further illustrates the membrane 150 extending along the inner surface of the filaments 142 a distance L5. Comparing the example stent embodiment of shown in FIG. 4 to that shown in FIG. 5 illustrates that the stent embodiment shown in FIG. 5 may include a relatively greater percentage of non-covered stent filaments 142 and relatively lower percentage of a membrane 150 covered stent scaffold. In some instances, the folded-over portion 160 may extend along and surround substantially the entire length of the uncovered portion of the medial region 126.

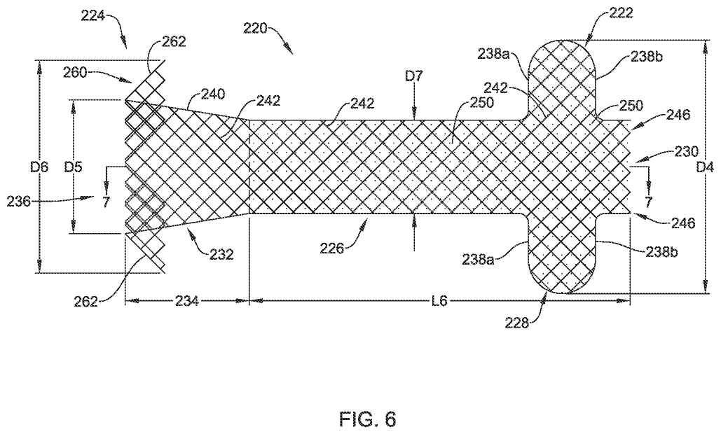

FIG. 6 illustrates an example expandable medical device, namely a stent 220 (e.g., drainage stent). The stent 220 may be similar in form and function to the stent 120 described with respect to FIG. 2. For example, the stent 220 may include a first end region 222 (e.g., a proximal end region), a second end region 224 (e.g., distal end region) and a medial region 226 extending between the first end region 222 and the second end region 224. The stent 220 may include one or more stent filaments 242 forming a tubular scaffold. Stent filaments 242 may extend helically, longitudinally, circumferentially, or otherwise along stent 220. While FIG. 6 shows stent filaments 242 extending along the entire length of stent 220, in other examples, the stent filaments 242 may extend only along a portion of stent 220.

In some examples, the stent 220 may be a self-expanding stent. Self-expanding stent examples may include stents having an expandable scaffold. In some instances, the self-expanding stent may have one or more filaments 242 combined to form a rigid and/or semi-rigid tubular stent scaffold. For example, the filaments 242 of the stent 220 may include wires or filaments which are braided, wrapped, intertwined, interwoven, weaved, knitted, looped (e.g., bobbinet-style) or the like to form the tubular scaffold. For example, while the example stents disclosed herein may resemble a braided stent, this is not intended to limit the possible stent configurations. Rather, the stents depicted in the Figures may be stents that are braided, knitted, wrapped, intertwined, interwoven, weaved, looped (e.g., bobbinet-style) or the like to form the stent scaffold. In various embodiments, the woven, braided and/or knitted member(s) may include a single filament woven upon itself, or multiple filaments woven together. In various embodiments, any of the woven, braided and/or knitted member(s), which comprise the elongate tubular body, may include a variety of different cross-sectional shapes (e.g., oval, round, flat, square, etc.).

Alternatively, the stent 220 may be a monolithic structure formed from a cylindrical tubular member, such as a single, cylindrical tubular laser-cut Nitinol tubular member, in which the remaining portions of the tubular member form the tubular scaffold of the stent 220. Openings or interstices through the wall of the stent 220 may be defined between adjacent filaments 242 or strut members.

The stent 220 in the examples disclosed herein may be constructed from a variety of materials. For example, the stent 220 (e.g., self-expanding or balloon expandable) may be constructed from a metal (e.g., Nitinol, Elgiloy, etc.). In other instances, the stent 220 may be constructed from a polymeric material (e.g., PET). In yet other instances, the stent 220 may be constructed from a combination of metallic and polymeric materials. Additionally, stent 220 may include a bioabsorbable and/or biodegradable material.

Additionally, the stent 220 may be configured to shift between a first (e.g., constrained, collapsed, non-expanded) configuration and a second (e.g., non-constrained, expanded) configuration. In an expanded configuration, the first end region 222 of the stent 220 may include a retention member 228 defining a first opening 230. The retention member 228 may be formed from the stent filaments 242 used to form other portions of the stent 220. For example, the retention member 228 may be formed from the same stent filaments 242 used to form the medial region 226.

FIG. 6 further illustrates that, in an expanded configuration, the second end region 224 of the stent 220 may include a flared portion 232 having a second opening 236. Additionally, similar to that described with respect to the stent 120 of FIG. 2, the second end region 224 of the stent 220 may be everted on itself to form a folded-over portion 260. For example, it can be appreciated that to form the folded-over portion 260, the stent scaffold may be wrapped, intertwined, interwoven, weaved, looped such that the individual filaments 242 extend continuously along the medial region 226 to the second end region 224 of the stent 220, whereby they curve and extend back (e.g., bend back) toward the first end region 222 of the stent 220. It can be appreciated that prior to being bent back over on themselves, the individual filaments 242 which form the folded-over portion 260 may have an inner surface which faces the longitudinal axis of the stent 220. However, in the folded-over configuration, the inner facing surface of the filaments 242 (which form the folded-over portion 260) have been everted and subsequently face outward away from the longitudinal axis of the stent 220. This first outwardly facing surface of filaments 242 is identified by reference numeral 262 in FIG. 6. It can be appreciated that this first outwardly facing surface 262 may extend circumferentially around the longitudinal axis of the stent 220. Additionally, FIG. 6 illustrates that the folded-over portion 260 may have a maximum outer diameter D6.

Additionally, the medial region 226 of the stent 220 may include a circumference and a longitudinal axis. The medial region 226 of the stent 220 may extend between the flared portion 232 of the second end region 224 and the retention member 228 of the first end region 222. The stent 220 may define an open interior lumen (e.g., passage, channel, etc.) extending from the first end region 222 to the second end region 224.

The retention member 228 may extend radially away from (e.g., substantially perpendicular to) the longitudinal axis of the medial region 226 to define a first surface 238a and a second surface 238b. In some instances, the first surface 238a, which may be a distally facing surface of the retention member 228, is substantially parallel to the second surface 238b, which may be a proximally facing surface of the retention member 228. The first surface 238a may be configured to atraumatically engage a (e.g., inner) tissue wall of a first body lumen (e.g., the stomach or duodenum). Further, as will be discussed in greater detail herein, the flared portion 232 (e.g., flared flange structure) of the second end region 224 may include an outer surface 240, a portion of which may be configured to atraumatically engage a (e.g., inner) tissue wall of an adjacent or apposed second body lumen (e.g., a biliary duct). In the example stent 220 illustrated in FIG. 6, the surfaces 262, 238a, 240 may prevent or limit movement/migration of the deployed stent 220 within or between the first and second body lumens.

FIG. 6 illustrates that, in some examples, an outer diameter D4 of the retention member 228 may be greater than the outer diameter D5 of the flared portion 232 and the outer diameter D6 of the folded-over portion 260. However, in other examples, the outer diameter D4 of the retention member 228 may be equal to the outer diameter D5 of the flared portion 232 and/or equal to the outer diameter D6 of the folded-over portion 260. In yet other examples, the outer diameter D4 of the retention member 228 may be less than the outer diameter D5 of the flared portion 232 and/or less than the outer diameter D6 of the folded-over portion 260. The medial region 226 may include a constant outer diameter D7 extending between the flared portion 232 and the retention member 228, whereby the diameter D7 of the medial region 226 is less than the diameters D4, D5 and D6 of the retention member 228, the flared portion 232, and the folded-over portion 260, respectively. As discussed herein, in some examples the outer diameter D7 of the medial region 226 may be substantially equal to the outer diameter D5 of the flared portion 232.

In some examples, the diameter D4 may be about 5 mm to about 40 mm, or about 10 mm to about 35 mm, or about 15 mm to about 30 mm, or about 20 mm to about 25 mm, or about 10 mm to about 20 mm, or about 20 mm to about 35 mm. In some examples, the diameter D5 may be about 2 mm to about 40 mm, or about 6 mm to about 30 mm, or about 10 mm to about 25 mm, or about 15 mm to about 20 mm, or about 6 mm to about 20 mm, or about 15 mm to about 35 mm. In some examples, the diameter D6 may be about 5 mm to about 40 mm, or about 10 mm to about 35 mm, or about 15 mm to about 30 mm, or about 20 mm to about 25 mm, or about 10 mm to about 20 mm, or about 20 mm to about 35 mm. In some examples, the diameter D7 may be about 2 mm to about 20 mm, or about 4 mm to about 18 mm, or about 6 mm to about 14 mm, or about 8 mm to about 12 mm, or about 6 mm to about 14 mm, or about 15 mm to about 25 mm.

Additionally, in some examples, the first end region 222 of the stent 220 may include free ends 246 of the one or more woven, braided or knitted strut members 242 which are not connected, and instead form sharp or pointed free ends of the strut members 242. As will be understood by those of skill in the art, the surface 238a of the retention member 228 may atraumatically engage an inner tissue wall of a first body lumen such that the free ends 246 extend into the first body lumen and do not contact the tissue wall.

As discussed herein, for HGS patients, stent migration may cause serious complications including death. If there is no tissue ingrowth-based adhesion at various anatomical regions, a deployed stent may migrate proximally into the stomach, causing leakage of biliary contents into the peritoneum, resulting in peritonitis. If there is sufficient or excessive adhesion at the hepatic duct, the stent may migrate distally into the peritoneum, causing leakage of biliary and stomach contents into the peritoneum, also causing peritonitis. Additionally, a migrated stent is free to abrade the outer gastric wall and other organs or vessels in the vicinity. Anatomically, stent migration can occur as a result of the hepatic duct being a generally static vessel, whereas the stomach is a highly motile vessel, as discussed herein. Accordingly, one method to reduce stent migration may include exposing bare metal portions of the stent to the tissue of the body lumen. The stent scaffold may then provide a structure that promotes tissue ingrowth (e.g., a hyperplastic response) into the interstices or openings thereof. The tissue ingrowth may anchor the stent in place and reduce the risk of stent migration.

FIG. 6 illustrates that, in some examples, the tubular scaffold of the stent 220 may include one or more non-covered (e.g., bare) portions designed to promote tissue ingrowth (e.g., a hyperplastic response) into the interstices or openings thereof. The non-covered portions may be devoid of a membrane, coating or other covering and thus the interstices of the tubular scaffold in the non-covered portions are open to tissue ingrowth. For example, FIG. 6 illustrates that the stent 220 may include the non-covered portion 234. It can be appreciated that, in some examples, the non-covered portion 234 of the tubular scaffold of the stent 220 may include the flared portion 232 and the folded-over portion 260 of the stent 120. In other words, the flared portion 232 and the folded-over portion 260 of the stent 220 shown in FIG. 6 may be bare (e.g., devoid or free of a membrane, coating, etc.) allowing tissue ingrowth through the interstices of the tubular scaffold along the flared portion 232.

Additionally, FIG. 6 illustrates that the stent 220 may include a membrane 250 (e.g., coating, membrane coating, etc.) extending along the tubular scaffold of the stent 220, such as within the lumen of the tubular scaffold of the stent 220. For example, FIG. 6 illustrates that the membrane 250 may extend along a length L6 of the tubular scaffold of the stent 220. The length L6 along which the membrane 250 extends within the lumen of the tubular scaffold of the stent 220 may include a portion of the medial region 226 and the first end region 222 (including the retention member 228). The membrane 250 may extend along inner surface and/or outer surface of the filaments 142 forming the medial region 226 in order to span across the interstices of the medial region 226, whereby the membrane 250 defines a leak-free, passageway (e.g., channel, lumen, tunnel, etc.) which may permit drainage of bodily substances (e.g., bile) from one anatomical organ (e.g., liver) to another anatomical organ (e.g., stomach). In some examples, the membrane 250 be an elastomeric or non-elastomeric material. For example, the membrane 250 may be a polymeric material, such as silicone, polyurethane, UE, PVDF, PTFE, ePTFE, ChronoFlex® or similar biocompatible polymeric formulations.

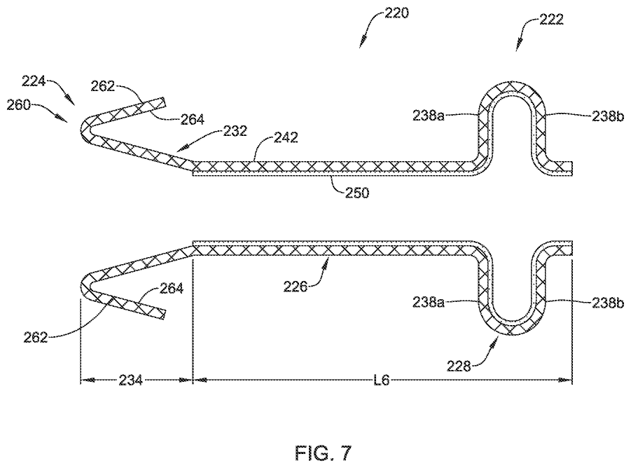

It can be further appreciated that the stent 220 may be designed to include regions which do not include the membrane 250 and regions which include the membrane 250 in different configurations than that illustrated in FIG. 6. In other words, the length of the non-covered portion 234 and the covered portion L6 may differ from that illustrated in FIG. 6. For example, the non-covered portion 234 may be longer than that shown in FIG. 6 (e.g., the non-covered portion may extend to a portion or all of the medial region 226). In other examples, more of the stent 220 may include a membrane 250. This would correspond to a lengthening of the length L6, whereby the membrane 250 may extend onto the flared portion 232. It can be appreciated that as the proportion of the stent 220 which includes a membrane 250 increases, the proportion of the stent 220 which does not include a membrane decreases, and vice versa. FIG. 7 illustrates a cross-sectional view of the stent 220 taken along line 7-7 of FIG. 6.

FIG. 7 illustrates the first end region 222, the second end region 224 and the medial region 226. The stent 220 may be formed from one or more braided, knitted, wrapped, intertwined, interwoven, weaved, looped (e.g., bobbinet-style) filaments 242 to form the tubular scaffold of the stent 220. FIG. 7 also illustrates the flared portion 232 and the folded-over portion 260 positioned along the second end region 224. The folded-over portion 260 may include the first outer facing surface 262 described herein. FIG. 7 further illustrates that the folded-over portion 260 may also include a second inward facing surface 264. The inward facing surface 264 may be defined as the surface of the folded-over portion 260 which faces toward the flared portion 232 and the longitudinal axis of the stent 220. Additionally, the stent 220 may include a retention member 228 positioned along the first end region 222.

FIG. 7 further illustrates the membrane 250 extending along a portion of the inner surface of the filaments 242 defining the tubular scaffold of the stent 220. In other words, FIG. 7 illustrates that the stent 220 may include the membrane 250 extending within the inner lumen of the tubular scaffold of the stent 220. In other instances, the membrane 250 may extend along a portion of the outer surface of the filaments 142 defining the tubular scaffold of the stent 220. As discussed herein, FIG. 7 illustrates the membrane extending along a length L6 of the medial region 226 of the stent 220. Further, FIG. 7 illustrates the non-covered portion 234 (including the folded-over portion 260). As discussed herein, the first outer-facing surface 262 and the second inner-facing surface 264 of the folded-over portion 260 may be devoid of the membrane 250, thereby permitting tissue ingrowth (e.g., a hyperplastic response) into the interstices or openings thereof. The entire non-covered portion 234 may be devoid of the membrane 250, coating or other covering and thus the interstices of the tubular scaffold in the non-covered portion 234 may be open to tissue ingrowth.

In some examples, the ratio of the covered portion L6 to the non-covered portion may be about 9:1 of the covered portion L6 to the uncovered portion 234, or about 4:1 of the covered portion L to the uncovered portion 234, or about 7:3 of the covered portion L6 to the uncovered portion 234, or about 3:2 of the covered portion L6 to the uncovered portion 234, or about 1:1 of the covered portion L6 to the uncovered portion 234. The uncovered portion 234 may be designed to be more flexible than the uncovered portion 234, allowing for better response when positioned in highly motile regions of the body. It can be appreciated that designing the stent 220 to include a relatively greater length of the uncovered portion 234 to the covered portion L6 may dedicate a greater percentage of the overall stent length to bare region duct drainage and anti-migration features. However, while longer uncovered portions 234 may allow for higher potential for side branch drainage and anti-migration ingrowth, the reduction in the covered portion L6 may reduce the effective length of the stent 220 that can be bridged between disconnected anatomical vessels (e.g., the distance between the gastric wall into the liver parenchyma). The uncovered portion 234 of the stent 220 must be sized to allow enough resistance to stent migration (so as to prevent migration initially through mechanical resistance and eventually with the addition of tissue ingrowth) while also allowing the stent 220 to include enough of a covered portion L6 to bridge the distance between disconnected anatomical vessels (e.g., the distance between the gastric wall into the liver parenchyma). Like the discussion set forth herein with respect to FIGS. 2-3, it can be appreciated that the stent 220 shown in FIG. 6 may be customized based on the where in the body the stent 220 may be positioned. For example, based on the rate of tissue ingrowth and/or side branch fluid flow within a particular organ (e.g., pancreas, hepatic duct, etc.), it may be desirable to design the stent 220 to have a shorter or longer fold-over portion 260 and/or more or less of the medial region 226 and/or the flared portion 232 to be covered by the membrane 250.

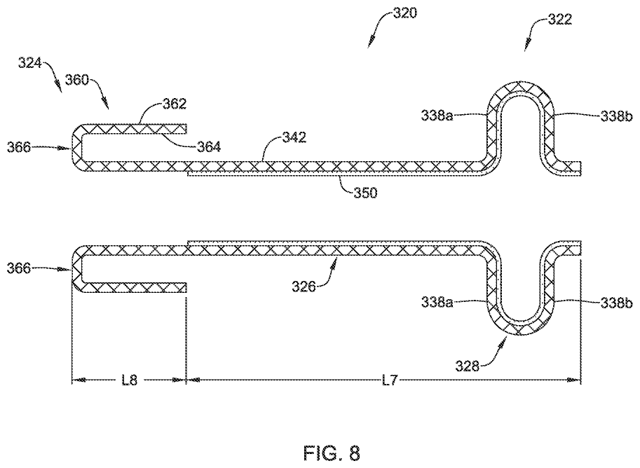

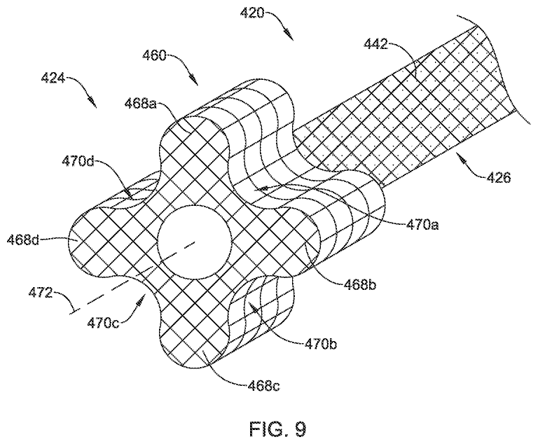

FIGS. 8-9 illustrate example stents that may be similar in form and function to the stents 120, 220 described above. For example, each of the stents shown in FIGS. 8-9 may include a membrane disposed along the tubular scaffold of the stent, such as within the lumen of the tubular scaffold of the stent (e.g., as described with respect to FIG. 2 and FIG. 6). The stents illustrated in FIGS. 8-9 may include various portions of the tubular scaffold of the stent which are uncovered to promote tissue ingrowth therethrough.

FIG. 8 illustrates a cross-sectional view of an example expandable medical device, namely a stent 320. The example stent 320 may be similar in form and function to other stents described herein. For example, the stent 320 may include a first end region 322, a second end region 324 and a medial region 226 extending therebetween. The stent 320 may be formed from one or more braided, knitted, wrapped, intertwined, interwoven, weaved, looped (e.g., bobbinet-style) filaments 342 to form the tubular scaffold of the stent 320. The stent 320 may also include a folded-over portion 360 positioned along the second end region 324.

Additionally, the stent 320 may include a retention member 328 positioned along the first end region 322. The retention member 328 may extend radially away from (e.g., substantially perpendicular to) the longitudinal axis of the medial region 326 to define a first surface 338a and a second surface 338b. In some instances, the first surface 338a, which may be a distally facing surface of the retention member 328, is substantially parallel to the second surface 338b, which may be a proximally facing surface of the retention member 328. The first surface 338a may be configured to atraumatically engage a (e.g., inner) tissue wall of a first body lumen (e.g., the stomach or duodenum).

FIG. 8 further illustrates that the stent 320 may include a membrane 350 extending along a portion of the inner surface of the strut members 342 defining the tubular scaffold of the stent 320. In other words, FIG. 8 illustrates that the stent 320 may include a membrane 350 extending along a length L7 within the inner lumen of the tubular scaffold of the stent 320, whereby the membrane 350 may be attached along the inner surface of the tubular scaffold of the stent 320.

FIG. 8 further illustrates that the folded-over portion 360 may include a substantially flat distal-facing surface 366 located at a distal extent of the stent 320. In some instances, the flat distal-facing surface 366 may line in a plane generally perpendicular to the central longitudinal axis of the stent 320. Additionally, similar to that described with respect to the stents 120, 220, the folded-over portion 360 may further include an outer facing surface 362 and an inner facing surface 364. Additionally, like the stent embodiment shown in FIG. 5, the outer facing surface 362 and the inner facing surface 364 of the folded-over portion 360 may be substantially parallel to the central longitudinal axis of the stent 320. In some examples, it can be appreciated that the outer facing surface 362 may extend circumferentially around the longitudinal axis of the stent 220 and, together with the distal facing surface 366, may define a substantially cylindrical shape. FIG. 8 illustrates that the folded-over portion 360 may extend toward the proximal end region 322 from the distal-facing surface 366 a length L8. The folded-over portion 360 may extend over and surround a portion of the medial region 326 of the stent 320 with the inner facing surfaced 364 spaced apart from the outer surface of the medial region 326 to define an annular gap therebetween. The folded-over portion 360, as well as the portion of the medial region 326 in which the folded-over portion 360 surrounds, may be devoid of a membrane or covering (e.g., bare), retaining interstices of the tubular scaffold open for tissue ingrowth and/or fluid drainage therethrough.

FIG. 9 illustrates a cross-sectional view of an example expandable medical device, namely a stent 420. FIG. 9 illustrates an alternative shape for the folded-over portion 460 along the distal end region 424 of an example drainage stent 420. Similar to other stent designs described herein, the stent 420 may be formed from one or more braided, knitted, wrapped, intertwined, interwoven, weaved, looped (e.g., bobbinet-style) strut members 442 to form the tubular scaffold and folded-over portion 460 of the stent 420.

FIG. 9 illustrates that the folded-over portion 460 may include one or more lobes 468a, 468b, 468c, 468d extending away from a central longitudinal axis 472 of the stent 420. FIG. 9 further illustrates that the folded-over portion 460 may also include one or more valleys 470a, 470b, 470c, 470d formed between two lobes 468a, 468b, 468c, 468d. It can be appreciated that the outer diameter (as measured from the central longitudinal axis 472) of the valleys 470a, 470b, 470c, 470d may be less than the outer diameter of the lobes 468a, 468b, 468c, 468d.

FIG. 9 further illustrates that the one or more lobes 468a, 468b, 468c, 468d may be spaced substantially equidistant around the central longitudinal axis 472 of the stent 420. Thus, the folded-over portion 460 may have an undulating outer and/or inner surface forming the lobes 468a, 468b, 468c, 468d and the valleys 470a, 470b, 470c, 470d. It can be appreciated that one advantage of the shape of the folded-over portion 460 (including the alternating lobes 468a, 468b, 468c, 468d and valleys 470a, 470b, 470c, 470d) is that the larger outer diameter of the lobes 468a, 468b, 468c, 468d may engage the tissue initially to provide early ingrowth opportunities while the smaller outer diameters of the valleys 470a, 470b, 470c, 470d, which would initially be spaced away from the tissue wall, maintain patency for continued drainage. Further, the lobed configured of the folded-over portion 460 may be incorporated into the folded-over portion of any of the other stents disclosed herein, including stents 120, 220, 320.

As discussed herein, the examples of stents described herein may be designed to permit leak-free drainage from one anatomical structure (e.g., hepatic ducts) to another anatomical structure (e.g., stomach) while also permitting tissue ingrowth into the stent to prevent stent migration. For example, FIG. 10 illustrates the folded-over portion 160 of the example stent 120 positioned in a hepatic duct 116 of the liver 106. FIG. 10 also illustrates the retention member 128 positioned in the stomach 102 with a laterally extending surface of the retention member 128 juxtaposed with the wall of the stomach 102.

As discussed herein, the uncovered portions of the stent 120 may encourage tissue ingrowth, which can be desirable in order to prevent migration of stent 120 after it has been appropriately positioned within the body. For example, configuration of the example stents disclosed herein may allow for resistance to migration based on the selective allowance of tissue ingrowth along uncovered portions of the stent. For example, the atraumatic surface 138 (shown contacting the inner wall 118 of the stomach 102) of the retention member 128 and also the surface along the outer facing surface 162 of the stent 120 may allow for resistance to migration based on the selective allowance of tissue ingrowth along those portions. As described herein, other stent designs disclosed herein may include additional regions for the allowance of tissue ingrowth. Additionally, FIG. 10 illustrates that the stent 120 may permit the flow of bile (or other body fluid) from the haptic duct 116 to the stomach 102 via the lumen of the stent 120 covered by the membrane 150. Furthermore, fluid drainage may be permitted through the interstices of the uncovered medial region of the stent 120 into the lumen of the stent 120.

The example stents shown in FIGS. 2-9 may contemplate braided stent designs which are utilized to permit leak-free drainage from one anatomical structure (e.g., hepatic ducts) to another anatomical structure (e.g., stomach) while also permitting tissue ingrowth into the stent to prevent stent migration. Additional stent designs are contemplated to permit leak-free drainage from one anatomical structure (e.g., hepatic ducts) to another anatomical structure (e.g., stomach) while also permitting tissue ingrowth into the stent to prevent stent migration. For example, knitted stents described herein may be utilized for the same purposes as the stents described in FIGS. 2-9.

It can be appreciated that for any of the example stent configurations described herein, coating and/or encapsulating a greater proportion of the stent strut members may result in a less flexibility stent design. In other words, the example stents disclosed herein illustrate stent designs having different proportions (e.g., ratios) of uncovered (e.g., bare) verses covered (e.g., with a membrane, coating, etc.) stent strut members. It can be appreciated that these designs may vary in flexibility, with the stents having a lower percentage of covered stent strut members being more flexible.

Further, any of the stent designs described herein may include a variety of different braid patterns, some of which may be relatively more or less “dense” than other braid patterns. For example, any of the stent designs described herein may include braid patterns with different wire counts, wire thicknesses or braid patterns. Example braid patterns may include a standard 2-over-2 configuration or a standard 1-over-1 configuration. Different braid patterns may result in different braid pattern density, stent flexibility, stent foreshortening characteristics, etc. It can be appreciated that different braid patterns may be utilized to implement different balances between tissue ingrowth into the interstices of the stent and/or drainage within the non-covered portions of the stent.

It can be appreciated that, in addition to the HGS procedure described herein, the example stents described herein may be used in other medical procedures. For example, the stent designs described herein may be utilized in a gastrojejunal (GJ) bypass procedure. One GJ procedure involves inserting one end of a stent into the stomach wall and the other end of the stent into a distal portion of the small intestine. The stent creates an anastomosis between the small intestine and the stomach, effectively rerouting the stomach contents directly into the small intestine. As both the stomach wall and intestine experience peristaltic motion, this would be considered a highly motile application, which may benefit from the stent, where the bypass can be achieved while allowing an adaptable channel which retains its diameter throughout peristalsis.

U.S. Provisional Patent Application No. 63/246,376, filed Sep. 21, 2021, U.S. patent application Ser. No. 17/941,867, filed Sep. 9, 2022, and U.S. Provisional Patent Application No. 63/427,618, filed Nov. 23, 2022, are herein incorporated by reference in their entirety for any and all purposes. These applications describe stents for implantation in body lumens and associated methods.

The stents, delivery systems, and the various components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic Nitinol; other nickel alloys such as nickel-chromium-molybdenum alloys, nickel-copper alloys, nickel-cobalt-chromium-molybdenum alloys, nickel-molybdenum alloys, other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys; platinum enriched stainless steel; titanium; combinations thereof; and the like; or any other suitable material.

Some examples of suitable polymers for the stents or delivery systems may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

In at least some embodiments, portions or all of the stents or delivery systems may also be doped with, made of, or otherwise include a radiopaque material. Radiopaque materials are generally understood to be materials which are opaque to RF energy in the wavelength range spanning x-ray to gamma-ray (at thicknesses of <0.005 inches (0.127 mm)). These materials are capable of producing a relatively dark image on a fluoroscopy screen relative to the light image that non-radiopaque materials such as tissue produce. This relatively bright image aids the user of the stents or delivery systems in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stents or delivery systems to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An expandable medical device, comprising:
a tubular scaffold including an inner surface, an outer surface, a proximal end region, a distal end region, and a medial region extending between the proximal end region and the distal end region, wherein the tubular scaffold defines a folded-over portion, and wherein the folded-over portion extends from the distal end region toward the proximal end region; and
a membrane disposed along the at least a portion of the medial region of the tubular scaffold.

2. The medical device of claim 1, wherein the folded-over portion extends away from the outer surface of the medial region toward the proximal end region at an acute angle.

3. The medical device of claim 2, wherein the folded-over portion includes interstices extending from an outer surface of the folded-over portion to an inner surface of the folded-over portion, and wherein the interstices are configured to permit tissue to grow therein.

4. The medical device of claim 3, wherein the folded-over portion extends circumferentially around a longitudinal axis of the tubular scaffold.

5. The medical device of claim 1, wherein the folded-over portion is positioned substantially parallel to a central longitudinal axis of the stent.

6. The medical device of claim 1, wherein the membrane extends along an inner surface of the medial region of the tubular scaffold from the distal end region to the proximal end region.

7. The medical device of claim 1, wherein the membrane extends along a portion of an inner surface of the medial region of the tubular scaffold, and wherein a portion of the medial region is devoid of the membrane.

8. The medical device of claim 7, wherein the portion of the medial region which is devoid of the membrane is positioned radially interior of to the folded-over portion.

9. The medical device of claim 1, wherein the membrane is configured to maintain a passageway for fluid to flow therethrough.

10. The medical device of claim 9, wherein the membrane is formed from an elastic material.

11. The medical device of claim 1, wherein the medical device further comprises a retention member extending radially away from the outer surface at the proximal end region, wherein the retention member has a distally facing surface positioned substantially parallel to a proximally facing surface.

12. The medical device of claim 11, wherein the tubular scaffold includes interstices extending from the outer surface of the tubular scaffold to the inner surface of the tubular scaffold, and wherein the membrane spans the interstices of the portion of the tubular scaffold which defines the retention member.

13. The medical device of claim 12, wherein the membrane is in direct contact with the inner surface of the portion of the tubular scaffold defining the retention member.

14. The medical device of claim 13, wherein the distal end region of the tubular scaffold further includes a flared portion.

15. The medical device of claim 14, wherein the folded-over portion is spaced apart from and extends away from the outer surface of the flared portion at an acute angle.

16. The medical device of claim 15, wherein the flared portion includes interstices extending from the outer surface of the tubular scaffold to the inner surface of the tubular scaffold, wherein the flared portion is devoid of the membrane such that tissue is permitted to grow through the interstices of the tubular scaffold along the flared portion.

17. The medical device of claim 16, wherein the retention member has an outermost diameter, wherein the flared portion has an outermost diameter, wherein the folded-over portion has an outermost diameter, and wherein the outmost diameter of the retention member is greater than the outmost diameter of the flared portion, the folded-over portion or both the flared portion and the folded-over portion.

18. An expandable medical device, comprising:
a tubular scaffold including an inner surface, an outer surface, a proximal end region, a distal end region, and a medial region extending between the proximal end region and the distal end region, wherein the tubular scaffold defines an everted portion, and wherein the everted portion extends from the distal end region toward the proximal end region;
a membrane disposed along a first portion of the medial region of the tubular scaffold, wherein a second portion of the medial region of the tubular scaffold is devoid of the membrane, and wherein the second portion of the medial region of the tubular scaffold which is devoid of the membrane is positioned radially interior to the everted portion.

19. The medical device of claim 18, wherein the everted portion extends away from the outer surface of the medial region at an acute angle.

20. An expandable medical device, comprising:
a tubular scaffold including an inner surface, an outer surface, a proximal end region, a distal end region, and a medial region extending between the proximal end region and the distal end region, wherein the tubular scaffold defines a folded-over portion, and wherein the folded-over portion extends from the distal end region toward the proximal end region; and
a membrane disposed along a first portion of the medial region of the tubular scaffold, wherein a second portion of the medial region of the tubular scaffold is devoid of the membrane, and wherein the second portion of the medial region of the tubular scaffold which is devoid of the membrane is positioned radially interior to and spaced apart from the folded-over portion;
wherein the tubular scaffold further comprises a retention member extending radially away from the medial region, wherein the retention member is a double-walled flange having a distal wall positioned substantially parallel to and spaced apart from a proximal wall of the double-walled flange.
